(19) United States
(12) Patent Application Publication
(10) Pub. No.: US 2025/0314568 A1
(43) Pub. Date: Oct. 9, 2025

(54) DEVICE FOR DETERMINING THE PEEL FORCE REQUIRED TO PEEL A COVER FOIL OFF OF A PRIMARY PACKAGING CONTAINER

(71) Applicant: Alcon Inc., Fribourg (CH)

(72) Inventors: Felix Brinckmann, Roßdorf (DE); Marcus Heift, Aschaffenburg (DE); Daniel Kessler, Lützelhausen (DE); Nils Schweizer, Bad König (DE)

(21) Appl. No.: 19/171,667

(22) Filed: Apr. 7, 2025

Related U.S. Application Data
(60) Provisional application No. 63/575,985, filed on Apr. 8, 2024.

(51) Int. Cl. G01N 3/04; G01N 19/04

(52) U.S. Cl. CPC G01N 3/04; G01N 19/04; G01N 2203/0091

(57) ABSTRACT

An automated device for determining the peel force to peel a lidding foil off of a top surface of a primary packaging shell comprises:
a shell holder for holding the primary packaging shell to the top surface of which the lidding foil is sealed;
a foil lifter for lifting a non-sealed end portion of the lidding foil up from the planar top surface of the primary packaging shell held by the shell holder;
a foil gripper for grasping the lifted non-sealed end portion of the lidding foil;
a foil peeler for peeling the sealed lidding foil with the lifted non-sealed end portion grasped by the foil gripper off of the planar top surface of the primary packaging; and
a force sensor for determining the force required to peel the lidding foil off of the top surface of the primary packaging shell.

### FIELD OF THE INVENTION

The present invention relates to a device for determining the peel force required to peel a cover foil off of primary packaging container. In particular, the primary packaging container may contain an ophthalmic article, for example an ophthalmic lens such as a contact lens, in particular a soft contact lens.

### BACKGROUND

In mass production of contact lenses, for example soft contact lenses which are worn only once and are disposed of after use, the soft contact lenses—once produced—are stored in so-called primary packaging containers that comprise a primary packaging shell, typically a plastic shell, in the bowl of which a storage liquid may be contained and in which the soft contact lens is stored. A lidding foil is sealingly attached, typically through heat-sealing or ultrasonic welding (alternatively through gluing), to the planar top surface of the plastic shell along a seal seam to form the closed and sealed primary package. A plurality of such closed and sealed primary packaging containers each containing a soft contact lens of the same type may then be packed in a secondary package (e.g. made of carton, or the like) for shipping.

To open a primary packaging container in order to get access to the soft contact lens stored in the storage liquid contained in the bowl, the lidding foil must be peeled off of the planar top surface of the plastic shell by the user, and subsequently the soft contact lens may be taken out of the bowl. While the lidding foil must be sealingly attached to the planar top surface of the plastic shell sufficiently strong to keep the bowl closed during storage, it must also allow the user to peel the foil off upon the application of peel forces that are within a certain predefined range so that the user may conveniently open the primary packaging container and get access to the soft contact lens.

In the statistic process control performed during production, samples of the closed and sealed primary packaging containers containing the soft contact lens stored in the storage liquid in the bowl of the plastic shell are taken from the production line and are examined remote from the production line. This examination includes the inspection of the soft contact lens as to its optical parameters, cosmetic defects, etc. However, for performing the statistic process control the primary packaging container must be opened first to get access to the soft contact lens. To achieve this, the lidding foil is peeled off of the planar top surface of the plastic shell manually by an operator, and then the lens is taken out from the bowl and examined. During peeling the lidding foil off of the planar top surface of the plastic shell, an assessment is also made by the operator as to whether or not the peel force required is acceptable. Obviously, this assessment is subjective as the process of peeling the foil off is performed by a human operator, even though the operator may be experienced. However, in addition to the assessment being subjective the process is time-consuming and expensive as it is performed by a human operator.

The overall number of soft contact lenses produced is constantly increasing, as is the number of different vision corrections (including toric vision corrections) required to optimally correct the various different visual defects (including astigmatism) of the users. While the number of different visual defects is increasing, the number of users having the same visual defect is decreasing. In terms of production of soft contact lenses, this means that the number of different lots of soft contact lenses to be produced is increasing while the size of the individual lots is decreasing. On the other hand, for the statistic process control it is vital that a representative number of samples are taken from each individual lot produced.

Obviously, therefore, the overall expense for performing the statistic process control is increasing, and this holds, too, for the expense related to the assessments as to whether or not the required peel force is acceptable, in addition to the latter assessments being subjective as they are performed by human operators.

### SUMMARY OF THE INVENTION

The present invention overcomes these disadvantages by suggesting an automated device for determining the peel force required to peel the lidding foil off of the primary packaging shell device as it is specified by the features of the independent claims. Advantageous aspects of the device according to the invention are the subject of the dependent claims.

In particular, the present invention suggests an automated device for determining the peel force required to peel a lidding foil off of a planar top surface of a primary packaging shell to which the lidding foil is sealed to form a primary packaging container, in particular a primary packaging container containing an ophthalmic article, for example an ophthalmic lens such as a contact lens, in particular a soft contact lens.

The device comprises:
a shell holder for holding the primary packaging shell to the top surface of which the lidding foil is sealed;
a foil lifter for lifting a non-sealed end portion of the lidding foil up from the planar top surface of the primary packaging shell held by the shell holder;
a foil gripper for grasping the lifted non-sealed end portion of the lidding foil;
a foil peeler to which the foil gripper is mounted, for peeling the sealed lidding foil with the lifted non-sealed end portion grasped by the foil gripper off of the planar top surface of the primary packaging shell to which it is sealed; and
a force sensor for determining the force required to peel the lidding foil off of the planar top surface of the primary packaging shell to which it is sealed.

According to one aspect of the device according to the invention, the shell holder comprises a shell support having shell receiving and positioning members for determining a position and a rotational orientation of the primary packaging shell on the shell support with the planar top surface of the primary packaging shell and the lidding foil sealed thereto facing upwards, and for preventing translational or rotational movement of the primary packaging shell on the shell support.

According to a further aspect of the device according to the invention, the shell receiving and positioning members comprise a suction channel arranged in the shell support for applying vacuum through the suction channel to make the primary packaging shell adhere to the shell support.

In accordance with another aspect of the device according to the invention, the foil gripper comprises a tweezer gripper with clamping jaws for grasping the lifted non-sealed end portion of the lidding foil and clamping it with the clamping jaws.

According to still a further aspect of the device according to the invention, the foil peeler comprises
a linearly movable slide to which the foil gripper is mounted, the linearly movable slide being linearly movable in a predetermined longitudinal direction in a plane that extends parallel to a plane of the planar top surface of the primary packaging shell with the lidding foil sealed thereto when the primary packaging shell is arranged on the shell support, the predetermined longitudinal direction of movement of the linearly movable slide corresponding to the direction in which the lidding foil is to be peeled off of the primary packaging shell arranged on the shell support at the position and with the rotational orientation determined by the shell receiving and positioning members; and
a linear motor having an armature that is linearly movable in the same longitudinal direction in which the linearly movable slide is linearly movable.

The force sensor is mounted to a proximal end of the foil gripper and to a distal end of the armature of the linear motor so that the force required to peel the lidding foil off of the top surface of the primary packaging shell is applied in the predetermined longitudinal direction by the armature of the linear motor through the force sensor to the foil gripper which is mounted to the linear slide.

According to yet a further aspect of the device according to the invention, the foil lifter for lifting the non-sealed end portion of the lidding foil up from the top surface of the primary packaging shell comprises a pin mounted to a vertically movable actuator.

The device of the present invention offers a number of advantages. First of all, the device is an automated device. That is, peeling the lidding foil off of the planar top surface of the primary packaging shell is performed automatically rather than by human operators. Accordingly, peeling the lidding foil off is repeatable a large number of times under the same conditions, and each time the required force for peeling the lidding foil off is objectively determined in an automated manner with the aid of a force sensor. Thus, any subjective assessment of the required force for peeling the lidding foil off by a human operator is avoided. Also, as no human operator is required anymore the expense for peeling the lidding foil off a number of times is substantially decreased.

The shell holder of the automated device may be any holder that is suitable to firmly hold the primary packaging shell in position when the lidding foil sealed to the planar top surface thereof is peeled off of the planar top surface. While in some embodiments the shell holder may comprise a shell support on which the primary packaging shell is arranged, in other embodiments the shell holder may comprise a mechanical clamp which may clamp the primary packaging shell at the front end and at the back end of the shell similar to how a user holds the shell when peeling the lidding foil off manually.

As will be seen from the detailed description of embodiments further below, the lidding foil is sealed to the top surface of the primary packaging shell along a seal seam that completely surrounds the bowl of the primary packaging shell to sealingly close the bowl containing the storage liquid and the contact lens contained therein. However, there are other portions, for example a front-end portion, of the lidding foil which are not sealed to the top surface of the primary packaging shell. The foil lifter lifts one such non-sealed end portion, for example the afore-mentioned front-end portion, of the lidding foil to allow a foil gripper to grasp this lifted non-sealed portion of the lidding foil.

Various types of foil lifters are conceivable. For example, the foil lifter may comprise a vacuum gripper arranged above the non-sealed end portion of the lidding foil through which suction is applied to lift the non-sealed end portion of the lidding foil up from the planar top surface of the primary packaging shell. Alternatively, the foil lifter may comprise a nozzle which is arranged beneath the primary packaging shell at a location of a through-opening of the primary packaging shell. To lift the non-sealed end portion of the lidding foil, an air jet may then be ejected from the nozzle which is directed through the through-opening of the primary packaging shell. As a further alternative, the foil lifter may comprise a mechanical pin mounted to an actuator that is vertically movable so that the pin is moved through the through-opening of the primary packaging shell to lift the non-sealed end portion of the lidding foil up from the planar top surface of the primary packaging shell.

Yet further, various types of foil peelers are conceivable. Once the non-sealed end portion of the lidding foil is lifted by the foil lifter, the lidding foil must be completely removed (i.e. peeled off) from the planar top surface of the primary packaging shell. This may be achieved with a wedge which is introduced in the gap formed between the lifted non-sealed end portion of the lidding foil and the planar top surface of the primary packaging shell, and which is then moved along the planar top surface of the primary packaging shell, thus removing the lidding foil from the planar top surface by shearing the foil off of the planar top surface (along the seal seam). Alternatively, the foil peeler may comprise a cylindrical bar having a slit, into which the lifted non-sealed portion of the lidding foil must be introduced (like a thread through a needle eye). Thereafter, the cylindrical bar is rolled about its bar axis over the planar top surface of the primary packaging shell, thereby peeling the foil off of the planar top surface of the primary packaging shell (along the seal seam). Yet further, the peeler may comprise a linearly movable slide to which the foil gripper may be attached, as will be explained in more detail below.

Advantageously, the shell holder may comprise a shell support having shell receiving and positioning members for determining a position and a rotational orientation of the primary packaging shell on the shell support. That is to say, any primary packaging shell may be placed on the shell support only at a particular position and with a particular rotational orientation, and is prevented from being translationally or rotationally moved. Once being arranged on the shell support, the planar top surface of the primary packaging shell and the lidding foil sealed thereto face upwards. The primary packaging shell with the lidding foil sealed to the planar top surface of the shell is then held down (retained) on the shell support by any suitable means, whereupon the lidding foil is peeled off of the planar top surface of the shell.

For holding the shell down on the shell support during peeling the lidding foil off, the shell receiving and positioning members of the shell support may comprise a suction channel arranged in the shell support for applying vacuum through the suction channel to make the primary packaging shell adhere to the shell support. From a technical point of view, this is a very simple and reliable way to hold the primary packaging shell down on the shell support.

Although various types of grippers may be used for the foil gripper, in an advantageous embodiment the foil gripper comprises a tweezer gripper with clamping jaws. This is a technically simple embodiment for the foil gripper that is capable of reliably grasping and clamping the lidding foil and keeping it clamped between the clamping jaws during the process of peeling the lidding foil off of the planar top surface of the primary packaging shell.

In a more detailed advantageous embodiment, the foil peeler comprises a linearly movable slide to which the foil gripper with the clamping jaws is mounted. This linearly movable slide is linearly movable in a predetermined longitudinal direction, and this longitudinal direction extends in a plane that is parallel to a plane defined by the planar top surface of the primary packaging shell with the lidding foil sealed thereto when the primary packaging shell is arranged on the shell support. This predetermined longitudinal direction of movement of the linearly movable slide corresponds to the direction in which the lidding foil is to be peeled off of the primary packaging shell arranged on the shell support at the position and the rotational orientation determined by the shell receiving and positioning members. This direction in which the lidding foil is to be peeled off of the planar top surface of the primary packaging shell typically also corresponds to the direction of the longitudinal extension of the primary packaging shell.

In this embodiment, the foil peeler further comprises a linear motor having an armature that is linearly movable in the same longitudinal direction in which the linearly movable slide is movable. The force sensor is mounted to a proximal end of the foil gripper on one hand and to the distal end of the armature of the linear motor on the other hand. Or to say it in other words, the force sensor is arranged straight in the ‘chain of transmission of the peel force’ between the armature of the linear motor and the foil gripper. The peel force required to peel the lidding foil off of the planar top surface of the primary packaging shell is generated by the linear motor, and is then transmitted from the armature of the linear motor through the force sensor to the foil gripper that holds the non-sealed end of the lidding foil clamped between its jaws. As the foil gripper is mounted to the linearly movable slide, once the armature of the linear motor is moved and moves the foil gripper, the linearly movable slide to which the foil gripper is mounted is linearly moved (e.g. along a guiding rail in a manner as frictionless as possible) together with the foil gripper. As the force sensor is arranged directly in the chain of transmission of the peel force from the armature of the linear motor to the foil gripper, the force required to peel the lidding foil off of the planar top surface of the primary packaging shell can be directly determined by the force sensor.

The foil lifter for lifting the non-sealed end portion of the lidding foil up from the top surface of the primary packaging shell may comprise a pin mounted to a vertically movable slide. This is a very simple and reliable solution to mechanically lift the non-sealed end portion of the lidding foil so as to enable the foil gripper to grasp—and in the case of the tweezer gripper to clamp—the non-sealed end portion of the lidding foil for subsequently performing the peeling procedure.

It is noteworthy that the ophthalmic article contained in the primary packaging container is described in the following as being a contact lens, in particular a soft contact lens such as a soft contact lens that is worn only once and is then disposed of. However, this is by way of example only. It is conceivable as well that other types of ophthalmic articles be stored in the primary packaging container, such as films for wetting the surface of the human eye or for releasing a drug or other substance upon being placed on the eye.

### BRIEF DESCRIPTION OF THE DRAWINGS

Further advantageous aspects of the device according to the invention will become apparent from the following description of embodiments of the invention with the aid of the drawings in which:

FIG. 1 shows a primary packaging container comprising a primary packaging shell to which a portion of the lidding foil is still sealed while another portion of the lidding foil is already peeled off;

FIG. 2 shows a perspective view of an embodiment of the device according to the invention in an initial state, in which the primary packaging shell with a lidding foil sealed thereto is arranged on a shell support;

FIG. 3 shows a side view of the embodiment of the device according to the invention in the initial state (first state) shown in FIG. 2;

FIG. 4 shows a side view of the embodiment of the device of FIG. 2, in a second state in which a non-sealed end portion of the lidding foil has been lifted up for being grasped by a gripper;

FIG. 5 shows a sectional view of the embodiment of the device in the second state shown in FIG. 4 (left and right sides exchanged when compared to FIG. 4);

FIG. 6 shows a side view of the embodiment of the device of FIG. 2, in a third state in which the lifted non-sealed end-portion of the lidding foil has been grasped and clamped between the jaws of the gripper;

FIG. 7 shows a side view of the embodiment of the device of FIG. 2, in a fourth state in which the lidding foil has been nearly completely peeled off of the top surface of the primary packaging shell; and

FIG. 8 shows a side view of the embodiment of the device of FIG. 2, in a final state (fifth state) in which the peeling procedure is completed and the lidding foil has been completely removed from the top surface of the primary packaging shell.

### DETAILED DESCRIPTION OF EMBODIMENTS

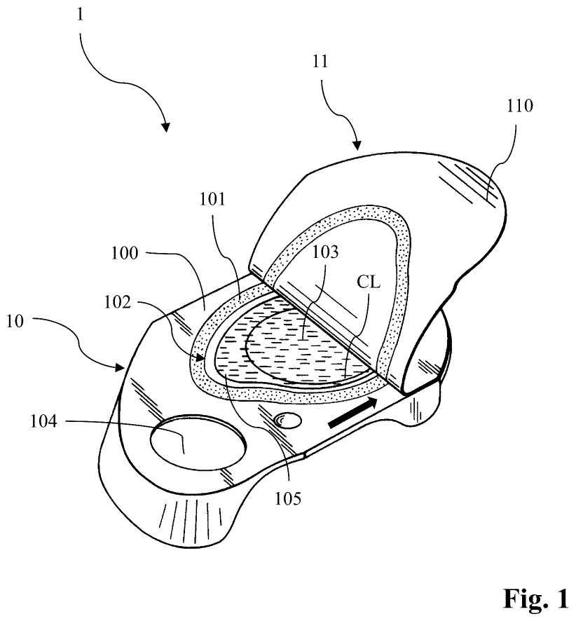

In FIG. 1 an embodiment of a primary packaging container 1 is shown as it is typically (but not exclusively) used for storing soft contact lenses which are used only once and are disposed of after use. Primary packaging container 1 comprises a primary packaging shell 10, typically made of a moldable plastic (for example polyethylene), and further comprises a lidding foil 11 sealed to a top surface 100 of primary packaging shell 10 along a seal seam 101 in the closed state of primary packaging container 1. In FIG. 1, primary packaging container 1 is shown in a state in which lidding foil 11 is partially peeled off of top surface 100 of primary packaging shell 10 so that a bowl 102 is visible that is filled with a storage liquid 103 and that contains a soft contact lens CL stored therein.

As can be seen in FIG. 1, when the lidding foil 11 is completely sealed to top surface 100 of primary packaging container 10 along seal seam 101, bowl 102 containing storage liquid 103 and soft contact lens CL is hermetically closed so that neither storage liquid 103 nor soft contact lens CL may escape from bowl 102, regardless of the position and orientation of primary packaging container 1. In order for a user to be able to open primary packaging container 1 to access soft contact lens CL, the user may grasp and hold the foremost downwardly protruding flange of primary packaging shell 10 with the thumb and the forefinger of one hand (e.g. the left hand) and slightly lift a non-sealed end portion 110 of lidding foil 11 up from the top surface 100 of primary packaging shell 10 by pushing the tip of the forefinger through through-opening 104 of primary packaging shell 10 from beneath. Thereafter, the user may grab the thus lifted non-sealed end portion 110 of lidding foil 11 with the thumb and the forefinger of the other hand (e.g. the right hand), clamp the non-sealed end portion 110 of lidding foil 11 therebetween, and peel lidding foil 11 off of top surface 100 of primary packaging shell 10 in the direction of the non-labeled arrow in FIG. 1 either partially, but preferably completely, while holding the downwardly protruding flange of primary packaging shell 10 with the thumb and forefinger of the one hand (e.g. the left hand). Once lidding foil 11 has been peeled off either partially or completely, the user may slide soft contact lens CL along a ramp 105 using the forefinger to take soft contact lens CL out and place it on the eye.

Obviously, the peel forces required to peel lidding foil 11 off of top surface 100 of primary packaging shell 10 should be within a predetermined range to make sure the user is able to conveniently peel lidding foil 11 off of top surface 100 on one hand, and on the other hand to make sure the lidding foil 11 is not inadvertently getting removed from top surface 100 (i.e. breaking the hermetical seal). During the process of examining samples of each lot of soft contact lenses produced on the production line as to their optical properties and other possible defects, primary packaging containers 1 are also examined with respect to the peel force required to peel lidding foil 11 off of top surface 100 of primary packaging shell 10.

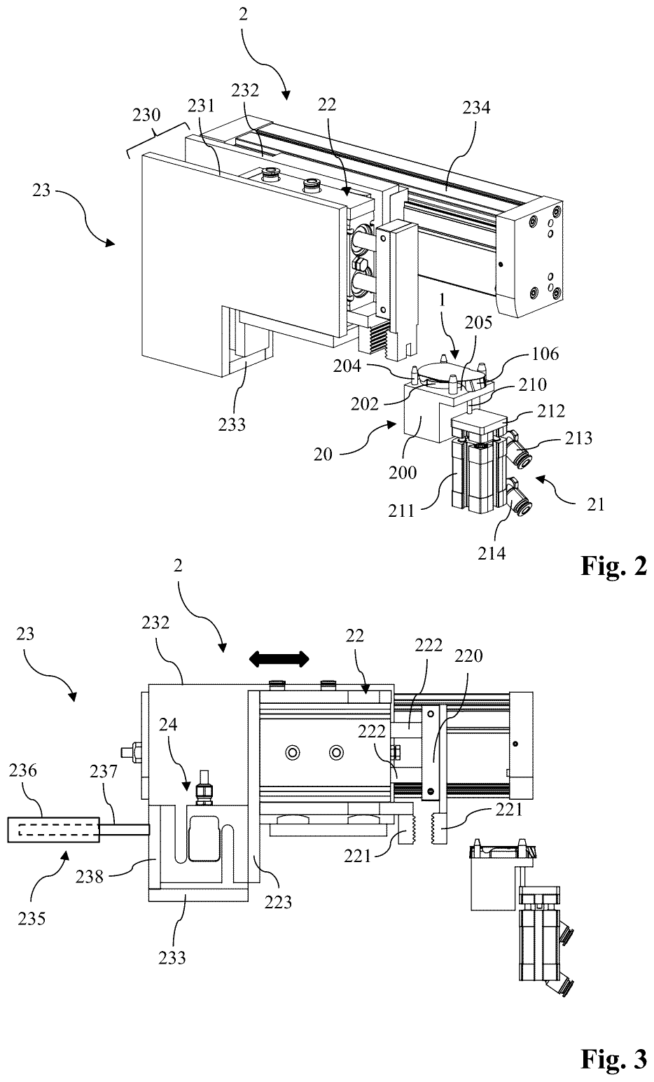

In FIG. 2 and FIG. 3 a perspective view and a side view of an embodiment of the automated device 2 of the present invention are shown in an initial state (first state). Device 2 comprises a shell holder 20 including a shell support 200 having shell receiving and positioning members. The shell receiving and positioning members determine the exact position and rotational orientation of the primary packaging shell 10 with the lidding foil 11 sealed thereto, i.e. of the sealed primary packaging container 1. The shell receiving and positioning members include a hollow receiving cylinder 202 for receiving the bowl 102 of primary packaging shell 10 upon being placed on the shell support 200. The contour of the upper surface of receiving cylinder 202 may correspond to the contour of the bowl 102 of primary packaging shell 10, and surrounds a cavity 201 (see FIG. 5) that forms the upper end of a suction channel 203 (see again FIG. 5) through which vacuum may be applied to make primary packaging shell 10 adhere to shell support 200. The shell receiving and positioning members further comprise four positioning pins 204 that project upwardly from a planar upper surface 205 of shell support 200 which may serve as a support surface for flange 106 of primary packaging shell 10. Accordingly, primary packaging shell 10 with lidding foil 11 sealed thereto may be placed on shell support 200 only at a predetermined position and with a predetermined rotational orientation, it is not possible to place it on shell support 200 at a different position or with a different rotational orientation.

Device 2 further comprises a foil lifter 21 for lifting non-sealed end portion 110 of lidding foil 11 up from planar top surface 100 of primary packaging shell 10. Foil lifter 21 comprises a pin 210 mounted to a vertically movable actuator 211 that comprises an upper platform 212 from which pin 210 projects upwardly. For example, vertically movable actuator 211 may be a pneumatic actuator having two connectors 213 and 214 for pressure supply conduits (not shown) in order to move upper platform 212 and thereby pin 210 up and down through the supply of pressure.

Still further, device 2 comprises a foil gripper 22 for grasping the non-sealed end portion 110 of lidding foil 11 once it is lifted. In the embodiment shown, foil gripper 22 comprises a tweezer gripper 220 with clamping jaws 221 for grasping the non-sealed end portion 110 of lidding foil 11 once it has been lifted by pin 210, and for clamping the non-sealed end portion 110 of lidding foil 11 between the clamping jaws 221.

Yet further, device 2 comprise a foil peeler 23 for peeling the sealed lidding foil 11 off of the planar top surface 100 of primary packaging shell 10 after the lifted non-sealed end portion 110 of lidding foil 11 has been grasped and clamped between the jaws 221 of tweezer gripper 220. To open and close the jaws 221 of tweezer gripper 220, an arm of tweezer gripper 220 may be pneumatically movable in the axial direction with the aid of two shafts 222.

Foil peeler 23 comprises a linearly movable slide 230 comprising a first lateral plate 231 (which is not shown in FIG. 3 for illustrative purposes) and a second lateral plate 232 which are connected to each by a bottom plate 233. Foil gripper 22 comprising tweezer gripper 220 is mounted in an almost frictionless manner between first lateral plate 231 and second lateral plate 232 with the aid of and guided by suitable bearings (not labelled in the drawings).

Linear slide 230 as a whole is movable along a fixedly arranged guiding rail 234 in a predetermined longitudinal direction extending in a plane that extends parallel to a plane of the planar top surface 100 of the primary packaging shell 10 with the lidding foil 11 sealed thereto. This predetermined longitudinal direction of movement of linearly movable slide 230 is indicated in FIG. 3 by the unlabeled double-headed arrow, and corresponds to the direction in which lidding foil 11 is to be peeled off of the planar top surface 100 of primary packaging shell 10 arranged on shell support 200 at the position and with the predetermined rotational orientation determined by the shell receiving and positioning members (i.e. receiving cylinder 202 and positioning pins 204). Prior to peeling the lidding foil 11 off, vacuum is supplied through the suction channel 203 (see FIG. 5) so that primary packaging shell 10 adheres to the shell support 200, as will be discussed in more detail below.

Foil peeler 23 further comprises a linear motor 235 (only shown in FIG. 3) having a stator 236 and an armature 237 that is linearly movable in the same longitudinal direction in which linear slide 230 is movable along fixedly arranged guiding rail 234. Stator 236 of linear motor 235 is also fixedly arranged, so that only the armature 237 is linearly movable.

A force sensor 24 is mounted to the distal end of the armature 237 of linear motor 235 by means of a first mounting plate 238 on one hand, and to the foil gripper 22 on the other hand by means of a second mounting plate 223. First mounting plate 238 is neither directly connected to the first lateral plate 231 nor to the second lateral plate 232 nor to the bottom plate 233 of linearly movable slider 230. Similarly, second mounting plate 223 is neither connected to any of the first lateral plate 231 or the second lateral plate 232 nor to the bottom plate 233. As mentioned, the force required to peel the lidding foil 11 off of the top surface 100 of primary packaging shell 10 is transmitted from the armature 237 through first mounting plate 238, through force sensor 24 and through second mounting plate 223 to foil gripper 22, and can therefore directly be determined by force sensor 24.

While FIG. 2 and FIG. 3 show the initial state (first state) of the device 2, in the following the full procedure of peeling the lidding foil 11 off of top surface 100 of primary packaging shell 10 is explained with the aid of additional states of device 2.

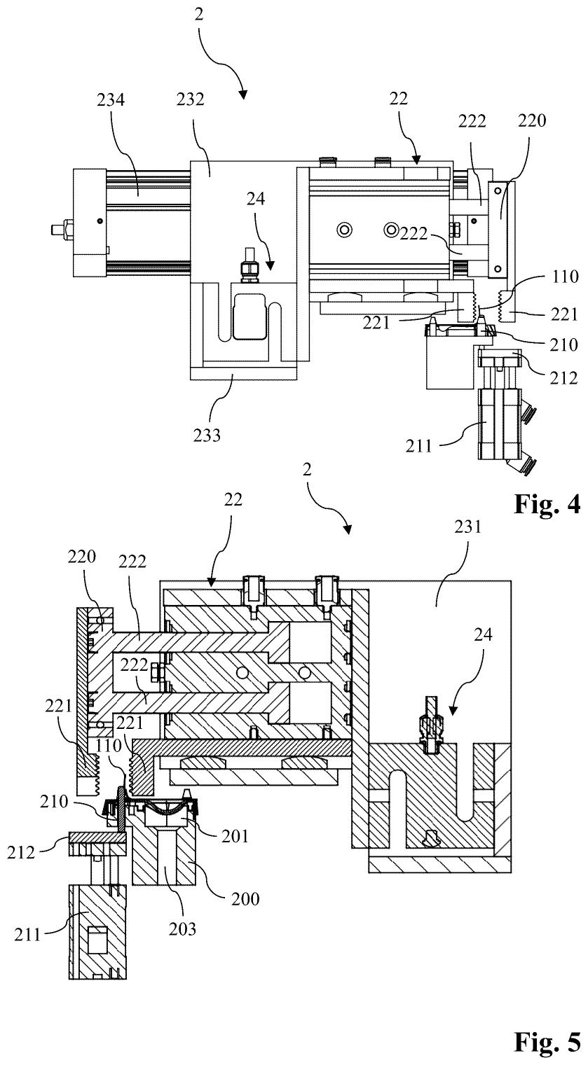

Accordingly, FIG. 4 and FIG. 5 show the device 2 in a second state in which the non-sealed end portion 110 of lidding foil 11 has been lifted (bent upwardly) by pin 210 mounted to vertically movable actuator 211 that comprises an upper platform 212 from which pin 210 projects upwardly. FIG. 4 shows a view similar to that of FIG. 3 (however, without linear motor 235), whereas FIG. 5 shows a sectional view in which the left and right sides of device 2 have been exchanged, and in which guiding rail 234 is not shown.

Foil gripper 22 together with linearly movable slide 230 (only second lateral plate 232 and bottom plate 233 visible in FIG. 4, only first lateral plate 231 and bottom plate 233 visible in FIG. 5) has been moved to a position in which the clamping jaws 221 of tweezer gripper 220 are arranged at a position ready for grasping the lifted non-sealed end portion 110 of lidding foil 11 and clamping it with the clamping jaws 221. As can be seen best in FIG. 5, the movable clamping jaw 221 may have a recess (non-hatched) that allows movable clamping jaw 221 to be moved (with the aid of shafts 222) towards the static clamping jaw 221 of tweezer gripper 220 to clamp the non-sealed end portion 110 of lidding foil 11 without colliding with the pin 210 that keeps the non-sealed end 110 of lidding foil 11 in the lifted state (i.e. bent upwardly).

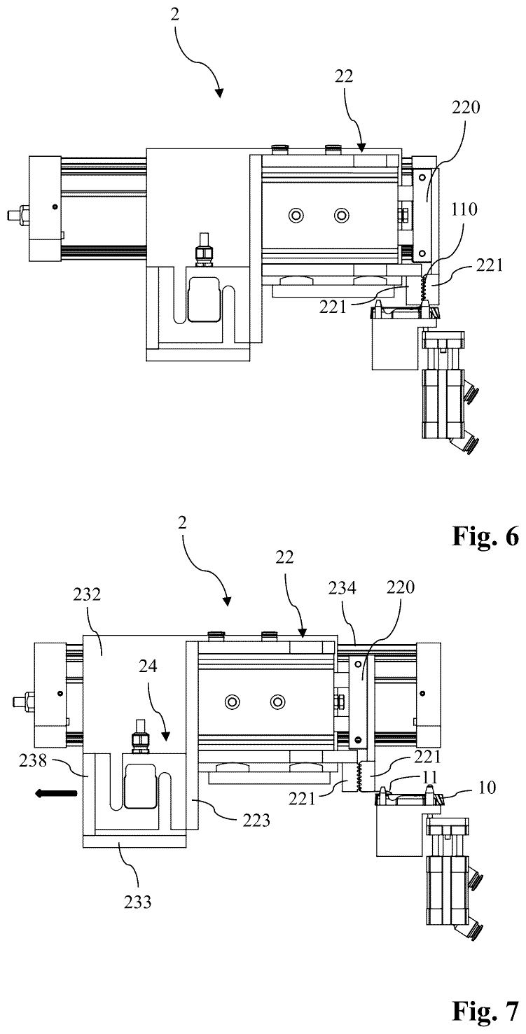

FIG. 6 shows device 2 (again without linear motor 235) in a third state in which clamping jaws 221 of tweezer gripper 220 are closed, and which the clamping jaws 221 clamp the non-sealed end portion 110 of lidding foil 11. Device 2 is now ready for peeling lidding foil 11 off.

FIG. 7 shows device 2 (again without linear motor 235) in a fourth state in which lidding foil 11 has been nearly completely peeled off of the top surface 100 of primary packaging shell 10. To reach this fourth state, the armature 237 of linear motor 235 (see FIG. 3) has applied a force (indicated by the unlabeled arrow in FIG. 7) to first mounting plate 238, and this force is transmitted through force sensor 24 to second mounting plate 223 and to foil gripper 22 comprising tweezer gripper 220 with the non-sealed end portion of lidding foil 11 clamped between the clamping jaws 221. The applied force peels lidding foil 11 off of top surface 100 of primary packaging shell 10 and can be determined by force sensor 24. Linearly movable slide (only second lateral plate 232 and bottom plate 233 thereof being visible in FIG. 7) to which foil gripper 22 is mounted is moved along guiding rail 234 with as low a friction as possible (preferably frictionless).

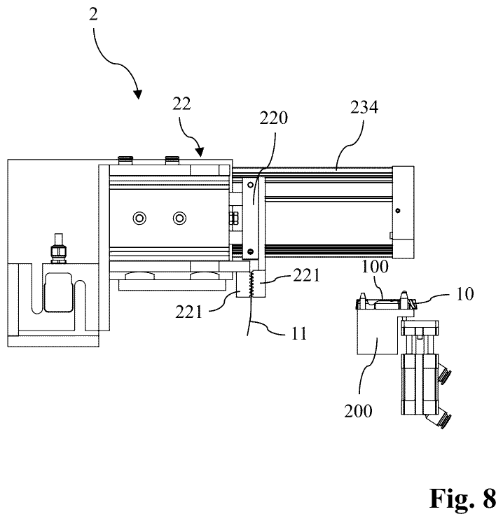

FIG. 8 shows device 2 (again without linear motor 235) in a final state (fifth state) in which lidding foil 11 has been completely peeled off of the top surface 100 of primary packaging shell 10, but with lidding foil 11 still being clamped by the clamping jaws 221 of tweezer gripper 220. Lidding foil 11 can then be placed in a foil waste bin (not shown). The storage liquid 103 and the soft contact lens CL are then removed from the bowl 102 of primary packaging shell 10, and the soft contact lens CL is subsequently inspected. Primary packaging shell 10 may be removed from shell support 200 and placed into a shell waste bin (not shown), thus being able to separate the different types of waste (e.g. for recycling purposes). Device may then be returned to the initial state (see FIG. 2) for the next sample (sealed primary packaging container) to be examined in the same manner.

While embodiments of the invention have been described with the aid of the drawings, it is apparent that many changes are possible without departing from the teaching underlying the invention. The scope of protection is therefore defined by the appended claims.

1. Automated device (2) for determining the peel force required to peel a lidding foil (11) off of a planar top surface (100) of a primary packaging shell (10) to which the lidding foil (11) is sealed to form a primary packaging container (1), in particular a primary packaging container containing an ophthalmic article, for example an ophthalmic lens such as a contact lens, in particular a soft contact lens (CL), the device (2) comprising:
a shell holder (20) for holding the primary packaging shell (10) to the top surface (100) of which the lidding foil (11) is sealed;
a foil lifter (21) for lifting a non-sealed end portion (110) of the lidding foil (11) up from the planar top surface (100) of the primary packaging shell (10) held by the shell holder (20);
a foil gripper (22) for grasping the lifted non-sealed end portion (110) of the lidding foil (11);
a foil peeler (23) to which the foil gripper (22) is mounted, for peeling the sealed lidding foil (11) with the lifted non-sealed end portion (110) grasped by the foil gripper (22) off of the planar top surface (100) of the primary packaging shell (10) to which it is sealed; and
a force sensor (24) for determining the force required to peel the lidding foil (11) off of the planar top surface (100) of the primary packaging shell (10) to which it is sealed.

2. Device according to claim 1, wherein the shell holder (20) comprises a shell support (200) having shell receiving and positioning members (202, 204) for determining a position and a rotational orientation of the primary packaging shell (10) on the shell support (200) with the planar top surface (100) of the primary packaging shell (10) and the lidding foil (11) sealed thereto facing upwards, and for preventing translational or rotational movement of the primary packaging shell (11) on the shell support (200).

3. Device according to claim 2, wherein the shell receiving and positioning members comprise a suction channel (203) arranged in the shell support (200) for applying vacuum through the suction channel (203) to make the primary packaging shell (10) adhere to the shell support (200).

4. Device according to claim 1, wherein the foil gripper (22) comprises a tweezer gripper (220) with clamping jaws (221) for grasping the lifted non-sealed end portion (110) of the lidding foil (11) and clamping it with the clamping jaws (221).

5. Device according to claim 2, wherein the foil peeler (23) comprises:
a linearly movable slide (230) to which the foil gripper (22) is mounted, the linearly movable slide (230) being linearly movable in a predetermined longitudinal direction in a plane that extends parallel to a plane of the planar top surface (100) of the primary packaging shell (10) with the lidding foil (11) sealed thereto when the primary packaging shell (10) is arranged on the shell support (200), the predetermined longitudinal direction of movement of the linearly movable slide (230) corresponding to the direction in which the lidding foil (11) is to be peeled off of the planar top surface (100) of the primary packaging shell (10) arranged on the shell support (200) at the position and with the rotational orientation determined by the shell receiving and positioning members (202, 204);
a linear motor (235) having an armature (237) that is linearly movable in the same longitudinal direction in which the linearly movable slide (230) is linearly movable;
and wherein the force sensor (24) is mounted to a proximal end of the foil gripper (22) and to a distal end of the armature (237) of the linear motor (235) so that the force required to peel the lidding foil (11) off of the top surface (100) of the primary packaging shell (10) is applied in the predetermined longitudinal direction by the armature (237) of the linear motor (235) through the force sensor (24) to the foil gripper (22) which is mounted to the linear slide (230).

6. Device according to claim 2, wherein the foil lifter (21) for lifting the non-sealed end portion (110) of the lidding foil (11) up from the top surface (100) of the primary packaging shell (10) comprises a pin (210) mounted to a vertically movable actuator (211).
